(19) United States
(12) Patent Application Publication
(10) Pub. No.: US 2007/0254013 A1
(43) Pub. Date: Nov. 1, 2007

(54) Medical Material and Process for Producing the Same

(75) Inventors: Tetsushi Taguchi, Ibaraki (JP); Hisatoshi Kobayashi, Ibaraki (JP); Junzo Tanaka, Ibaraki (JP); Kazuo Tsubota, Chiba (JP); Naoshi Shinozaki, Chiba (JP); Shigeto Shimmura, Chiba (JP); Hideyuki Miyashita, Chiba (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NAT'L INSTITUTE FOR MATERIALS SCIENCE, Tsukuba-shi (JP)

(21) Appl. No.: 10/588,309

(22) PCT Filed: Feb. 1, 2005
(86) PCT No.: PCT/JP05/01410

§ 371 Date: Jun. 4, 2007

(30) Foreign Application Priority Data
Feb. 4, 2004 (JP) 2004-028581

(51) Int. Cl. A61F 2/14

(52) U.S. Cl. 424/427

(57) ABSTRACT

[Object] A medical material that improves therapeutic effects in epithelial cells such as keratoconjunctival epithelial cells with the use of an amnion, and a process for producing the same are provided. [Solving Means] A medical material includes an amnion, which is a placental tissue, a polymer film bonded to one surface of the amnion and crosslinked, and cells adhered to the other surface of the amnion. A process for producing the medical material includes the steps of preparing an amnion from which the spongy layer is removed, bonding a biocompatible polymer film to one surface of the amnion followed by crosslinking, adhering epithelial stem cells to the other surface of the amnion, and proliferating epithelial cells from the epithelial stem cells on the surface of the amnion.

### TECHNICAL FIELD

The present invention relates to a medical material and a process for producing the same, and more specifically, to a medical material including an amnion to which a biocompatible polymer film is bonded and crosslinked, and a process for producing the same.

### BACKGROUND ART

The cornea is disposed at the outermost part of the optical system constituting the eyeball and is a clear tissue that does not contain blood vessels. The cornea and tear fluid form a smooth eyeball surface, thereby obtaining satisfactory eyesight. Keratoconjunctival epithelial cells are constantly in contact with the external world and have a function of protecting the eyeball from foreign matters such as microorganisms in the external world and rays of light such as ultraviolet rays. That is, the keratoconjunctival epithelial cells play an extremely important role in order to maintain the clarity of the cornea and to protect the whole eyeball to maintain homeostasis.

When the cornea becomes cloudy and loses its clarity because of a disease such as keratitis, corneal ulcer, or perforation of the cornea, eyesight is permanently degraded. As a therapy for the degradation of eyesight caused by such a clouding of the cornea, corneal transplantation is performed. In the corneal transplantation, a cornea of a patient from which the clarity is lost is removed, and a new clear cornea is transplanted. By performing such transplantation, clarity is recovered, and eyesight can be restored again.

The cornea is composed of five layers including, from the surface, an epithelium (about 50 μm in thickness), Bowman's membrane (about 10 μm), a stromal layer (about 500 μm), Descemet's membrane (about 10 μm), and an endothelial layer (about 5 μm). The cornea has a total thickness of 0.5 mm, and the stromal layer accounts for 90% of the thickness. A cornea to be transplanted must be clear and have a thickness of about 0.5 mm. A cornea having an uneven thickness is not satisfactory in view of strength and cannot provide satisfactory eyesight because such a cornea causes irregular astigmatism. The corneal epithelium covers the surface of the cornea and has functions of preventing bacteria from intruding and maintaining an optically smooth surface. Such a cornea suitable for transplantation is only a cornea derived from a donor.

When a cornea is not donated because of a shortage of donors, patients requiring a corneal transplantation including the stroma cannot undergo medical treatment. If only a polymer film is transplanted instead of the stroma, the transplanted film is removed and cannot be maintained for a long period of time. When a polymer film is transplanted, since the epithelium is not immobilized alive, the polymer film must be maintained without the epithelium. However, when the epithelium is not present, protection from the intrusion of bacteria cannot be achieved, resulting in an infection. As a method of transplanting only the corneal epithelium, a transplantation method using an amnion has been developed (Non-Patent Documents 1 and 2).

The amnion used in this transplantation method can be obtained from, for example, the placenta of pregnant women who have delivered by Caesarean section. In addition, since this amnion has a thick basement membrane, the amnion functions as a substrate for proliferation and differentiation of keratoconjunctival epithelial cells. Furthermore, the amnion hardly has immunogenicity, and the amnion has an anti-inflammatory effect, an effect of suppressing the formation of scars, and the like. Therefore, the keratoconjunctival epithelium or a stem cell tissue of the epithelium that is transplanted on the amnion can be protected from rejections by the recipient or the like.

In the transplantation method using an amnion, first, ocular scar tissue forming a part of the cornea is removed to expose the cornea and the sclera. An amnion is applied on the exposed cornea and sclera in order to reconstruct the ocular surface. The central part of a cornea (including the epithelium, the central layers, and the endothelium) is cut out from donated corneal tissue, and the periphery of the corneal limbus is trimmed. The donated corneal tissue is then transplanted and placed on the exposed amnion and the stroma. The corneal limbus thus transplanted is then differentiated and proliferated using the amnion as a substrate in a state in which the corneal limbus is protected by the amnion that has no immunogenicity. Thus, the corneal epithelium is regenerated on the amnion.

An improved method of reconstructing an ocular surface using an amnion involves applying epithelial stem cells on the amnion in advance and culturing them in vitro, and proliferating epithelial cells from the stem cells so that the epithelial cells cover the surface of the amnion (see Patent Documents 1 to 5).
Non-Patent Document 1: Medical Asahi, the September issue in 1999, pp. 62-65
Non-Patent Document 2: Kazuo Tsubota et al., N Engl J Med 340, pp. 1697-1703, 1999
Patent Document 1: Japanese Unexamined Patent Application Publication No. 2001-161353
Patent Document 2: Japanese Unexamined Patent Application Publication No. 2002-320666
Patent Document 3: Japanese Unexamined Patent Application Publication No. 2002-331025
Patent Document 4: Japanese Unexamined Patent Application Publication No. 2003-126236
Patent Document 5: PCT Japanese Translation Patent Publication No. 2003-532466

### DISCLOSURE OF INVENTION

Problems to be Solved by the Invention

In the methods of reconstructing an ocular surface using an amnion disclosed in the above patent documents, transplantation is performed using the whole amnion on which epithelial stem cells and epithelial cells proliferated from the epithelial stem cells are adhered.

However, a transplant in which such an amnion is used as a substrate has a thickness of less than 100 μm and does not have a layer corresponding to the stroma. Therefore, such a transplant is fragile in view of strength. Although such a transplant is suitable for reconstruction of an ocular surface, it is not suitable for transplantation that requires a transplant with a certain thickness including the stroma. In cases where the lesion extends to the stroma, a further improvement is desired in the transplantation method using an amnion.

It is an object of the present invention to provide a medical material that improves therapeutic effects in epithelial cells such as keratoconjunctival epithelial cells with the use of an amnion, and a process for producing the same.

Means for Solving the Problems

In order to achieve the above object, the present inventors have found that when epithelial stem cells are cultured on the surface of an amnion that is bonded and crosslinked with a biocompatible polymer film, a medical material including the biocompatible polymer film, the amnion, the epithelial stem cells, and epithelial cells proliferated from the epithelial stem cells so as to cover the amnion and to form a single layer can be obtained.

Namely, the present invention provides a material for reconstructing an ocular surface to be transplanted on an affected part where not only epithelial cells but also their stem cell tissue is eradicated or damaged, the material including an amnion, which is a placental tissue; a clear biocompatible polymer film bonded to one surface of the amnion and crosslinked with covalent bonds; and corneal epithelial cells, corneal stromal cells, corneal endothelial cells, or conjunctival epithelial cells that are adhered to the other surface of the amnion.

The present invention also provides a process for producing the material for reconstructing an ocular surface including the steps of preparing an amnion from which the spongy layer is removed, bonding a biocompatible polymer film to one surface of the amnion followed by crosslinking, adhering epithelial stem cells to the other surface of the amnion, and proliferating epithelial cells from the epithelial stem cells on the surface of the amnion.

### ADVANTAGES OF THE INVENTION

Since a biocompatible polymer film has high biocompatibility, after a transplant is transplanted, the survival rate is increased compared with the case where a biocompatible polymer film is not used. Furthermore, since a biocompatible polymer film is clear and the thickness of the film can be appropriately changed, the biocompatible polymer film is particularly effective for the transplantation of the corneal epithelium or the conjunctival epithelium and the stroma.

Furthermore, by culturing epithelial stem cells on an amnion that is bonded and crosslinked with a biocompatible polymer film in advance, advantageously, handleability is improved and suturing can be easily performed. The film can be applied to an artificial cornea by immobilizing the corneal epithelium alive on one side of the film and the corneal endothelium alive on the other side of the film.

### ADVANTAGES OF THE INVENTION

Since a biocompatible polymer film has high biocompatibility, after a transplant is transplanted, the survival rate is increased compared with the case where a biocompatible polymer film is not used. Furthermore, since a biocompatible polymer film is clear and the thickness of the film can be appropriately changed, the biocompatible polymer film is particularly effective for the transplantation of the corneal epithelium or the conjunctival epithelium and the stroma.

Furthermore, by culturing epithelial stem cells on an amnion that is bonded and crosslinked with a biocompatible polymer film in advance, advantageously, handleability is improved and suturing can be easily performed. The film can be applied to an artificial cornea by immobilizing the corneal epithelium alive on one side of the film and the corneal endothelium alive on the other side of the film.

### BEST MODE FOR CARRYING OUT THE INVENTION

Among embryonic membranes formed in the development process of Amniota (reptiles, birds, and mammals) of vertebrates, the amnion is the innermost membrane that directly covers an embryo. The amnion has a thick basement membrane, and a histocompatibility antigen is not exhibited on the surfaces of the cells thereof. Therefore, since the amnion hardly has immunogenicity and the amnion has an anti-inflammatory effect and the like, the amnion is a material suitable for transplantation. Although the amnion hardly has immunogenicity, in order to reliably prevent the occurrence of an immune reaction after transplantation and to prevent interspecific infection, an amnion derived from the same animal as the animal species to which the medical material of the present invention is transplanted is preferably used. That is, in order to produce a medial material for being transplanted to the human being, a human amnion is preferably used. As long as an amnion derived from the same animal species as that of an animal to which a transplant is transplanted is used, the amnion is not limited to amnions of those related by blood such as a parent or a child, but amnions of others can also be used.

Amnions derived from human beings can be obtained from pregnant women who have delivered by Caesarean section. Amnions obtained from women other than pregnant women who have delivered by Caesarean section may be generally contaminated, and thus such amnions are difficult to use. An amnion generally includes, from the bottom layer side, a spongy layer (stratum spongiosum), a compact layer, a basement membrane layer, and an epithelial layer. In the amnion used in the medical material of the present invention, the spongy layer and the epithelial layer are preferably removed in advance. Hereinafter, unless otherwise stated, the term “amnion” means an amnion from which the spongy layer and the epithelial layer are removed.

An amnion is bonded to a biocompatible polymer film and crosslinked. The biocompatible polymer film that can be used in the present invention may be a biopolymer, a synthetic polymer, or a hybrid of these polymers. Examples of the biopolymer include proteins, glycosaminoglycans, proteoglycans, alginic acids, chitosans, polyamino acids, and combinations of two or more of these. Examples of glycosaminoglycans include chondroitin sulfate, dermatan sulfate, hyaluronic acid, heparan sulfate, heparin, keratan sulfate, and derivatives thereof. Examples of proteins include collagen, atelocollagen, alkali-treated collagen, gelatin, keratin, serum albumin, ovalbumin, hemoglobin, casein, globulin, fibrinogen, and derivatives thereof.

Examples of the synthetic polymer used for the biocompatible polymer film include polymers of a water-soluble monomer, and water-soluble polymers. Examples of the water-soluble monomer include n-isopropylacrylamide, acrylamide, acrylic acid, methacrylic acid, and vinylpyrrolidone, and combinations of two or more of these. Examples of the water-soluble polymers include polyvinyl alcohol (PVA), polyallylamine, polyvinylamine, aliphatic or aromatic diisocyanates, PVA into which an amino group is introduced by CNBr, and combinations of two or more of these.

Among these, in view of the ease of availability and high affinity for the cornea or the like, collagen is preferably used. As a film of collagen, a crosslinked product of an alkali-treated I-type collagen is preferred. For example, an alkali-treated I-type collagen derived from pigskin available from NITTA GELATIN Inc. can be used as such a film. There are more than ten and less than twenty types of collagen, but the type and the origin of collagen are not limited.

An example of the form of a crosslinking bond between the amnion and the biocompatible polymer film is the immobilization with covalent bonds. Such an immobilization can be performed by a crosslinking process in the presence of a crosslinking agent such as glutaraldehyde or formaldehyde and collagen or gelatin. This process can be specifically performed through the following steps.

First, an amnion is spread over a glass plate, and a mixed solution of a crosslinking agent and collagen is applied on the amnion. A biocompatible polymer film is then placed thereon, thereby conducting a crosslinking reaction between the amnion and the biocompatible polymer film. Thus, the amnion can be immobilized. Alternatively, the process of forming a crosslinking bond can be performed by applying only the crosslinking agent on the amnion to react with the biocompatible polymer film. Since such a crosslinking bond is formed by crosslinking with covalent bonds, the resulting film has a feature that the amnion and the biocompatible polymer film are not separated even under physiological conditions and can be stably present.

Epithelial stem cells that are adhered on the amnion crosslinked with the biocompatible polymer film are cells having an ability of producing epithelial tissue cells by cell division. Examples of the epithelial cells used in the present invention include corneal epithelial cells and conjunctival epithelial cells. Stem cells of corneal epithelial cells that produce the corneal epithelium by cell division are present on the corneal limbus. Stem cells of conjunctival epithelial cells that produce the conjunctival epithelium by cell division are present on the conjunctival fornix and the like. These cells can be taken out from a living body by cutting out tissue of the region including the cells.

The removed cells can be adhered to an amnion by the following method. For example, the cut-out tissue may be placed directly on the amnion. Alternatively, the cut-out tissue may be treated with an enzyme such as trypsin, dispase, or the like to extract only the cells, and the cells may then be seeded on the amnion. The cells adhered on the amnion can be proliferated on the amnion, for example, in the presence of feeder layer cells using a culture medium prepared by adding 15 volume percent of serum, cholera toxin, an epidermal growth factor, insulin, and dimethyl sulfoxide to a DMEM/F12 liquid medium. Stratified epithelial cells as in the cornea of a living body can be obtained by proliferating the cells until the entire surface of the amnion is covered with the cells, and then culturing the proliferated cells in a state in which the liquid volume of the culture medium is decreased so that the cells are exposed to air.

The case where the corneal limbus is used as the epithelial stem cell tissue will be described, but the case where the conjunctival fornix or the like is used as the epithelial stem cells is similar. Similarly, corneal endothelial cells can also be used as the medical material of the present invention.

The epithelial stem cell tissue such as the corneal limbus or the conjunctival fornix including epithelial stem cells may be tissue that is substantially composed of only stem cells or tissue that includes not only stem cells but also epithelial cells, fibroblasts, and vascular endothelial cells. The epithelial stem cell tissue may be tissue obtained from a blood relation of a recipient who undergoes the transplantation or tissue obtained from others who have no blood relationship with the recipient. When epithelial stem cells are obtained from a person other than the recipient, in order to prevent a problem of rejection due to the immunity, epithelial stem cells derived from a donor who is compatible in HLA type are preferred. However, when epithelial stem cells derived from a donor who is compatible in HLA type cannot be obtained, epithelial stem cells derived from a donor who is not compatible in HAL type may be used. In order to prevent an infection due to the transplantation or the like, the donated tissue used is preferably confirmed in advance to be free of infection or the like. Epithelial stem cells obtained from recipients themselves who undergo the transplantation can be used as the epithelial stem cells as long as the cells are determined to include stem cells.

Furthermore, preferably, the epithelial stem cell tissue is in good condition and can proliferate epithelial cells. The size of the stem cell tissue cut out from a donor or recipients themselves may be, for example, about 1 mm square compared with an amnion having a size of 2 cm square when the condition of the stem cell tissue is good. The size of the stem cell tissue cut out can be appropriately changed according to the condition of the cells, the degree of the disease of the recipient, and the like.

The epithelial stem cell tissue cut out from a donor is adhered to the amnion bonded and crosslinked with the biocompatible polymer film. The epithelial stem cell tissue is preferably adhered to the amnion so that, when the medical material of the present invention is transplanted, the epithelial stem cell tissue is disposed at a position where the epithelial stem cell tissue is originally located in vivo. For example, when the epithelial stem cell tissue is the corneal limbus, preferably, the tissue is formed so as to substantially have the size of the cornea of the recipient and is disposed substantially in the shape of a ring.

The epithelial stem cells disposed on the amnion bonded and crosslinked with the biocompatible polymer film are immersed in a culture solution in vitro so that the amnion and the stem cells are immersed therein, and the stem cells are then exposed to air. Thereby, the epithelium can be stratified. The epithelial cells include cells obtained by proliferating the stem cells contained in the epithelial stem cell tissue by cell division. However, the epithelial cells may further include cells generated by cell division of epithelial cells other than the stem cells, the epithelial cells being contained in the donated epithelial stem cell tissue. Eventually, epithelial cells are proliferated and differentiated from the (epithelial) stem cells on the amnion so as to cover the surface of the amnion, and preferably, stratification of the epithelial cells is accelerated. Alternatively, instead of performing the proliferation in vitro, the epithelial stem cell tissue may be transplanted to a recipient in a state in which the epithelial stem cell tissue is adhered to the amnion, and thus the proliferation of epithelial cells may be performed on the affected part of the recipient.

The epithelial cells proliferated on the amnion are preferably in good condition when being transplanted. For example, preferably, the epithelial cells are cells in the logarithmic growth phase and in a state in which metabolic activity is steadily maintained and division is repeated at a constant cell cycle.

Thus, the medical material including an amnion, a biocompatible polymer film bonded and crosslinked with the amnion, an epithelial stem cell tissue adhered on a surface of the amnion, the surface not being bonded to the biocompatible polymer film, and epithelial cells proliferated from the stem cell tissue is transplanted on an affected part where not only epithelial cells but also their stem cell tissue is eradicated or damaged.

### EXAMPLE 1

1. Preparation of Biocompatible Films

1) Biopolymer Gel (Collagen Gel Sheet)

Glutaraldehyde was added to a dimethyl sulfoxide (DMSO) solution (30 weight percent) of collagen so that the final concentration of glutaraldehyde was 6 mM. The mixed solution was cast between two glass plates having a silicone rubber spacer with a thickness of 500 μm, and reaction was conducted at 37° C. for 24 hours to prepare a collagen gel sheet. Subsequently, the gel sheet was immersed in a significantly excessive amount of a phosphate buffer to replace DMSO.

2) Synthetic Polymer Gel (PVA Gel Sheet)

A water/DMSO mixed solvent solution (15 volume percent) of PVA (average degree of polymerization: 77,000) was heated in an autoclave at 120° C. for five hours to dissolve PVA. The solution was cast between two glass plates having a silicone rubber spacer with a thickness in the range of 200 to 500 μm. The solution was left to stand in a freezer at −20° C. for two hours, and was then thawed at room temperature to prepare a PVA gel sheet. Subsequently, the freeze-dried PVA gel sheet was placed in a 10 weight percent toluene solution of a tin catalyst-containing hexamethylenediisocyanate. The solution was then stirred at room temperature for one hour under nitrogen bubbling, thereby introducing isocyanato groups into the surfaces of the PVA gel sheet. The gel sheet was immersed in a significantly excessive amount of a phosphate buffer to replace the isocyanato groups with amino groups.

2. Chemical Immobilization of Amnion on Biocompatible Polymer Film

Glutaraldehyde serving as a crosslinking agent was added to a DMSO solution (30 weight percent) of collagen so that the final concentration of glutaraldehyde was 200 mM, and the mixed solution was dripped on an amnion. The mixed solution of collagen gel was then applied and the collagen gel sheet or the PVA gel sheet prepared in section 1 was placed thereon. The reaction of immobilization was conducted at 37° C. for 24 hours. As shown in the cross-sectional optical microscopy image (hematoxylin-eosin (HE) staining) of FIG. 1, an amnion-immobilized collagen gel was obtained.

3. Formation of Epithelial Cell Layer on Amnion Immobilized on Biocompatible Polymer Film

As feeder layer cells, 3T3 cells treated with mitomycin C were seeded on the lower well of Transwell. Subsequently, the limbus cut out from a human cornea for transplantation donated from an overseas donor was treated with a 0.05 weight percent trypsin-EDTA solution at 37° C. for one hour, thereby separating epithelial cells containing stem cells. The amnion-immobilized collagen gel sheet or the amnion-immobilized PVA gel sheet prepared in the process in section 2 was placed on the upper well of the Transwell so that the amnion was disposed on the upper side. Epithelial cells were seeded on the amnion and cultured until cells completely covered the surface of the amnion. In the stratification of the epithelial layer, the liquid volume of the culture medium was decreased so that the surfaces of the epithelial cells were exposed to air, and the cells were cultured for two weeks.

FIG. 2 shows an optical microscopy image (HE staining) of a cross-section in which corneal epithelial cells were proliferated by a stratified culture on the amnion-immobilized collagen gel sheet. FIG. 3 shows an optical microscopy image (HE staining) of a cross-section in which corneal epithelial cells were proliferated by a stratified culture on the amnion-immobilized PVA gel sheet. Each image shows that a uniform corneal epithelial layer having a thickness of about 50 μm was formed.

### BRIEF DESCRIPTION OF THE DRAWINGS

FIG. 1 is an optical microscopy image (hematoxylin-eosin (HE) staining) as a drawing showing a cross-section of an amnion-immobilized collagen gel of Example 1.

FIG. 2 is an optical microscopy image (HE staining) as a drawing showing a cross-section in which corneal epithelial cells are proliferated by a stratified culture on an amnion-immobilized collagen gel sheet by a method of Example 1.

FIG. 3 is an optical microscopy image (HE staining) as a drawing showing a cross-section in which corneal epithelial cells are proliferated by a stratified culture on an amnion-immobilized PVA gel sheet by the method of Example

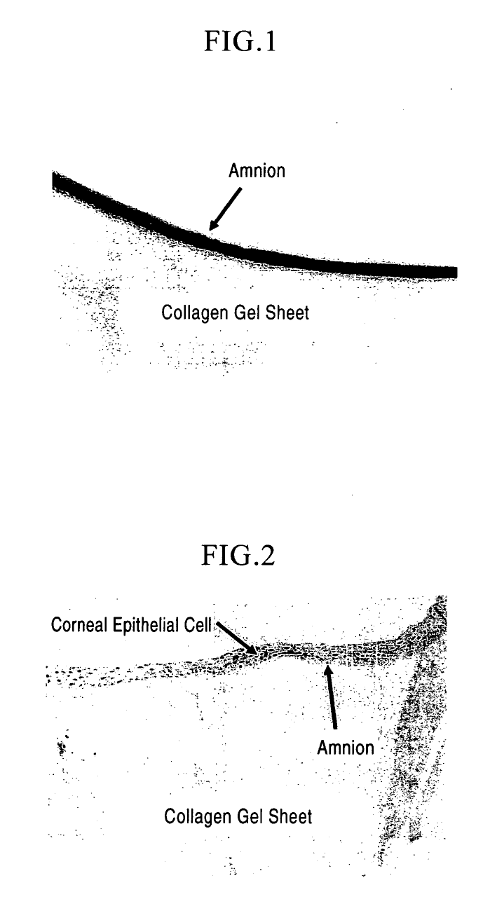

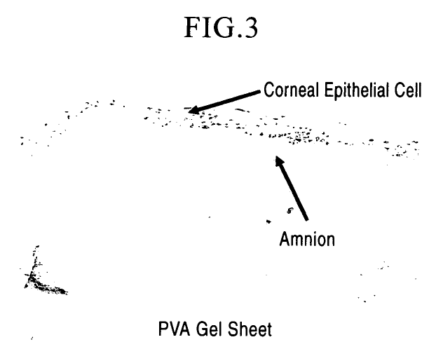

1. A material for reconstructing an ocular surface to be transplanted on an affected part where not only epithelial cells but also their stem cell tissue is eradicated or damaged, the material comprising an amnion, which is a placental tissue; a clear biocompatible polymer film bonded to one surface of the amnion and crosslinked with covalent bonds; and corneal epithelial cells, corneal stromal cells, corneal endothelial cells, or conjunctival epithelial cells that are adhered to the other surface of the amnion.

2. The material for reconstructing an ocular surface according to claim 1, wherein the cells described in claim 1 are epithelial cells that are proliferated and differentiated from stem cells on the amnion to be stratified.

3. (canceled)

4. The material for reconstructing an ocular surface according to claim 1 or 2, wherein the polymer film described in claim 1 is a gel composed of a biopolymer, a synthetic polymer, or a combination of two or more of these polymers.

5. A process for producing the material for reconstructing an ocular surface according to claim 1, comprising the steps of preparing an amnion from which the spongy layer is removed, bonding a biocompatible polymer film to one surface of the amnion followed by crosslinking, adhering epithelial stem cells to the other surface of the amnion, and proliferating epithelial cells from the epithelial stem cells on the surface of the amnion.
